(19) United States
(12) Patent Application Publication
(10) Pub. No.: US 2010/0143346 A1
(43) Pub. Date: Jun. 10, 2010

(54) Treatment of Macular Degeneration

(76) Inventors: Frances Margaret Richards, Oxfordshire (GB); Alan Geoffrey Roach, Oxfordshire (GB)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/520,715

(22) PCT Filed: Dec. 21, 2007
(86) PCT No.: PCT/GB07/04947

§ 371 Date: Feb. 19, 2010

(30) Foreign Application Priority Data
Dec. 22, 2006 (GB) 0625844.6

(51) Int. Cl. A61K 39/395; A61P 27/02; A61P 27/06; A61P 27/10; A61P 33/02; A61P 27/14; A61P 31/04; A61P 31/22; A61K 31/7088; A61K 31/575; A61K 31/57; A61K 38/12

(52) U.S. Cl. 424/133.1; 514/44 R; 514/170; 514/171; 514/179; 514/11

(57) ABSTRACT

An agent having progesterone antagonist properties may be used to treat eye conditions associated with pathological blood vessel formation, for example age-related macular degeneration, choroidal neovascularisation, retinal neovascularisation or corneal neovascularisation. The agent may be mifepristone.

### FIELD OF THE INVENTION

This invention relates to the treatment and prophylactic prevention of conditions associated with undesired blood vessel formation and in particular ocular conditions characterised by the presence of unwanted neovascularisation, such as neovascular age-related macular degeneration and diabetic retinopathy.

### BACKGROUND TO THE INVENTION

Retinal and choroidal neovascularization (CNV) can lead to hemorrhage and fibrosis, with resulting visual loss in a number of conditions of the eye, including, for example, age-related macular degeneration, ocular histoplasmosis syndrome, pathologic myopia, angioid streaks, idiopathic disorders, choroiditis, choroidal rupture, overlying choroid nevi, Best's disease, Stargardt's disease, Vogt-Koyanagi-Harada syndrome, toxoplasmosis, certain inflammatory diseases, sickle cell disease, diabetic retinopathy and other proliferative retinopathy, retinopathy of prematurity, neovascular glaucoma, sarcoidosis, syphilis, pseudoxanthoma elasticum, vein or artery occlusion, carotid obstructive disease, chronic uveitis/vitritis, mycobacterial infection, Lyme's disease, Eale's disease, systemic lupus erythomatosus, Behcet's disease, infections causing retinitis, optic pits, par planitis, chronic retinal detachment, hyperviscosity syndromes, capillary haemangioma including von Hippel-Lindau disease, trauma and post-laser complication.

In addition, pathological neovascularisation can occur in the cornea. Examples of corneal neovascularization include, epidemic keratoconjunctivitis, Vitamin A deficiency, contact lens overwear, atopic keratitis, superior limbic keratitis, pterygium keratitis sicca, sjogrens, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi sarcoma, Mooren ulcer, Terrien's marginal degeneration, mariginal keratolysis, rheumatoid arthritis, systemic lupus, polyarteritis, trauma, Wegeners sarcoidosis, Scleritis, Steven's Johnson disease, pemphigoid radial keratotomy, and corneal graft rejection.

One of the disorders, namely, age-related macular degeneration (AMD), is the leading cause of severe vision loss in people aged 65 and above (Leibowitz, Krueger et al. 1980; Klein and Klein 1982; Bressler, Bressler et al. 1988). Patients with early AMD have drusen and altered retinal pigmentation, with only subtle abnormalities in visual function. Patients with late stage AMD have severe vision loss due to either geographic atrophy (“dry” AMD) or CNV (“wet” AMD). In CNV, abnormal blood vessels originate from the choriocapillaris beneath the retina and grow under the retinal pigment epithelium (RPE) and retina. These new vessels have a tendency to leak, resulting in distortion of the macula and central vision loss.

Vascular Endothelial growth factor (VEGF) plays a key role in angiogenesis by stimulation of proliferation of endothelial cells and by permeabilisation of vessels. Other pro-angiogenic factors are also involved, including fibroblast growth factor, platelet derived growth factor, insulin-like growth factor, transforming growth factors alpha and beta, angiopoietin-1 and -2. There are also anti-angiogenic factors that play a role in controlling angiogenesis, including pigment epithelial derived growth factor (PEDF), thrombospondin, angiostatin, and endostatin. An imbalance in the ratio of angiogenic and anti-angiogenic factors, including increased VEGF and decreased PEDF, is thought to contribute to CNV. Induction of VEGF through the hypoxia-response element in the VEGF promoter is required for development of CNV in a mouse model (Muranaka, Yanagi et al. 2005; Vinores, Xiao et al. 2006) and it is thought that in AMD outer retina hypoxia may stimulate VEGF production to drive CNV formation (Schlingemann 2004).

The potential for anti-VEGF therapy to influence CNV (wet AMD) has been recognised with the development of several drugs designed to inhibit VEGF. The anti-VEGF aptamer Pegaptinib (Macugen) and the anti-VEGF antibodies ranibizumab (Lucentis) and bevacizumab (Avastin) have shown promise in clinical trials but these drugs require regular dosing by intravitreal injection, which is an invasive technique with risks of trauma and endophthalmitis (Yeoh, Sims et al. 2006). Thus, there is a market for anti-angiogenic agents that can be administered in a less invasive manner, ideally by topical administration, or perhaps using a slow release delivery system.

Progesterone receptor mRNA has been detected in many different ocular tissues (Suzuki, Kinoshita et al. 2001; Fuchsjager-Mayrl, Nepp et al. 2002), including rabbit retina/choroid (Wickham, Gao et al. 2000) and chick retina (Lippman, Wiggert et al. 1974) but the cell types in which the receptor is expressed has not been elucidated and the role of these receptors in the eye is not clear. The progesterone receptor (PR) was also demonstrated to function in intact chick retina (Li, Hayes et al. 1997). Progesterone may have a role in function of the neuroretina since it is present in the rat retina (Lanthier and Patwardhan 1988) and it enhances survival of rat retinal ganglion cells in culture (Lindsey and Weinreb 1994). Also, in isolated bovine retinal pigment epithelial (RPE) microsomes progesterone inhibits acyl CoA:retinol acyltransferase (ARAT) (Ross 1982; Kaschula, Jin et al. 2006), an enzyme which may be a component of the visual cycle.

The most commonly known progesterone receptor antagonist is RU486, i.e. 17beta-hydroxy-11-beta-(4-dimethylaminophenyl)-17alpha-(prop-1-ynyl)-estra-4,9-dien-3-one, also known as mifepristone, RU38486 and C-1073. RU486 competes with endogenous progesterone by high affinity competitive receptor binding. It also acts as a high affinity competitive inhibitor for the glucocorticoid receptor, binding with two to three times the affinity of dexamethasone (Moguilewsky and Philibert 1984; Gagne, Pons et al. 1985). RU486 is used clinically as an abortifacient and has been investigated extensively for use as a contraceptive (Baulieu and Ulmann 1986; Sitruk-Ware and Spitz 2003). It has also been tested in clinical trials for psychotic depression (Simpson, El Sheshai et al. 2005; Flores, Kenna et al. 2006), Alzheimer's disease (DeBattista and Belanoff 2005), schizophrenia (Gallagher, Watson et al. 2005), uterine myomas (Eisinger, Bonfiglio et al. 2005), endometriosis (Kettel, Murphy et al. 1996), leiomyoma (Fiscella, Eisinger et al. 2006), meningioma (Spitz, Grunberg et al. 2005) and hormone-dependent breast cancer (Perrault, Eisenhauer et al. 1996). RU486 shows a high level of oral bioavailability (40-70%). After oral dosing it reaches peak plasma levels after 1-2 hours, with a plasma half life of 20-30 hours. Concentrations in the blood are limited by the need for complexing with a specific carrier protein, with unbound mifepristone being metabolised in the liver. Metabolism involves demethylation and hydroxylation, with nine metabolites identified in the rat, four of which have also been detected in man. The three primary metabolites detected in man are an N-mono-demethylated compound (RU42633), N-didemethylated (RU42848) and a hydroxylated compound (RU42698). The fourth is an N-acetylated derivative of RU42848. The three primary metabolites show strong progesterone receptor and glucocorticoid receptor binding activity (Baulieu and Segal 1985).

RU486 has been administered topically to the eye of rabbits (Phillips, Green et al. 1984; Tsukahara, Sasaki et al. 1986), in order to test its ability to lower intraocular pressure due to its glucocorticoid receptor antagonist activity. Cheeks and Green (Cheeks and Green 1986) measured the concentration of RU486 in rabbit ocular tissues after administration of eye drops and showed that while the vitreous concentration was low, that of the retina and especially choroid were higher, suggesting that passage of the drug through the iris root into the suprachoroidal space and beyond occurs. Therapeutically relevant concentrations of active compound should reach the choroid and retina after topical administration of RU486 or related molecule (e.g. a metabolite of RU486) and as such the preferred embodiment is to have an eye drop formulation. However, therapeutically effective concentrations may also be achieved by oral administration (see infra).

It is possible that special drug delivery systems may be needed in some cases in order to achieve the required concentration at the retina and choroid. Examples of technology that might be employed to this end are sustained release micro or nano particles such as those used for retinal delivery of budesonide (Kompella, Bandi et al. 2003), ocular inserts, contact lenses, gel-to-sol systems, vesicular systems, liposomes, niosomes, mucoadhesive dosage forms, penetration enhancers, microemulsions, iontophoresis, dendrimers, all of which are reviewed by Sultana et al. (2006).

### SUMMARY OF THE INVENTION

The present invention is based at least in part on the realisation that a progesterone antagonist, e.g. a progesterone receptor modulator, may be used to prevent or treat pathogenic angiogenesis in the eye, especially VEGF-mediated choroidal neovascularisation. In this scenario, and without wishing to be bound by theory, a PR modulator delivered to the eye can reach sufficiently pharmacologically active PR antagonist concentrations at the back of the eye to impact on the level of PR activity. Reduction of the level of activity of the PR then reduces angiogenesis by modulation of VEGF. Thus, according to the present invention, a compound having PR antagonist activity is used for manufacture of a medicament for the treatment of ocular neovascularisation (in particular Age Related Macular Degeneration), wherein said compound possesses PR receptor binding activity that inhibits the agonist activity of progestins.

According to a first aspect, the invention provides the use of an agent having progesterone antagonist properties for the manufacture of a medicament for use in the treatment of a condition associated with blood vessel formation.

In preferred embodiments, the invention contemplates the use of mifepristone (RU486), or a metabolite or pharmaceutically acceptable derivative thereof, for the treatment of wet AMD. In such embodiments, the mifepristone (RU486) metabolite may be selected from RU42698, RU42848 and RU42633. Other preferred progesterone antagonists are onapristone and asoprisnil (see infra).

Also contemplated are pharmaceutical compositions for topical administration to the eye comprising mifepristone (RU486), or a metabolite or pharmaceutically acceptable derivative thereof. In such embodiments, the mifepristone (RU486) metabolite may be selected from RU42698, RU42848 and RU42633 The mifepristone (RU486), or metabolite or pharmaceutically acceptable derivative thereof, is preferably present in an amount sufficient to act as a PR antagonist on administration of the composition to the eye.

Also contemplated is an ocular drug delivery system (e.g. a slow release ocular drug delivery system) comprising mifepristone (RU486), or a metabolite or pharmaceutically acceptable derivative thereof. In such embodiments, the mifepristone (RU486), or metabolite or pharmaceutically acceptable derivative thereof, may be present in an amount sufficient to act as a PR antagonist on administration of the composition to the eye.

Other aspects of the invention are recited in the claims set out below.

### General Preferences and Definitions

References herein to any particular progesterone antagonist (and in particular references to particular progesterone receptor (PR) antagonists, e.g. RU486) include pharmaceutically acceptable derivatives (e.g. salts) and analogues (e.g. metabolites, such as the RU486 metabolites RU42633, RU42848 and RU42698) thereof. Thus, references herein to RU486 (mifepristone) are to be interpreted to cover RU486 metabolites and pharmaceutically acceptable salts thereof.

The term pharmaceutically acceptable derivative as applied to the progesterone antagonists of the invention define progesterone antagonists which are obtained (or obtainable) by chemical derivatization of the parent progesterone antagonists. The pharmaceutically acceptable derivatives are suitable for administration to or use in contact with mammalian tissues without undue toxicity, irritation or allergic response (i.e. commensurate with a reasonable benefit/risk ratio). Preferred derivatives are those obtained (or obtainable) by demethylation, hydroxylation, acetylation, alkylation, glycosidation, esterification or acylation of the parent progesterone antagonists. The derivatives may be active per se, or may be inactive until processed in vivo. In the latter case, the derivatives of the invention act as pro-drugs. Particularly preferred pro-drugs are ester derivatives which are esterified at one or more of the free hydroxyls and which are activated by hydrolysis in vivo. The pharmaceutically acceptable derivatives of the invention may retain some or all of the activity of the parent progesterone antagonist. In some cases, the activity is increased by derivatization. Derivatization may also augment other biological activities of the progesterone antagonist, for example bioavailability.

Moreover, in cases where the progesterone antagonist for use according to the invention is optically active, the present invention contemplates all optical isomers, racemic forms and diastereoisomers of the progesterone antagonist. Thus, references to any particular progesterone antagonist (e.g. to any particular PR antagonist) are to be interpreted to encompass racemic mixture of diastereoisomers, individual diastereoisomers, as a mixture of enantiomers as well as in the form of individual enantiomers.

A “pharmaceutical composition” is a solid or liquid composition in a form, concentration and level of purity suitable for administration to a patient (e.g. a human or animal patient) upon which administration it can elicit the desired physiological changes. Pharmaceutical compositions are typically sterile and/or non-pyrogenic. The term non-pyrogenic as applied to the pharmaceutical compositions of the invention defines compositions which do not elicit undesirable inflammatory responses when administered to a patient.

### DETAILED DESCRIPTION OF THE INVENTION

The antiprogestin (PR antagonist) activity of a compound for use in the invention can be determined by the in vitro functional assay (Edwards et al. (1995); Morgan et al. (2002) J. Med. Chem. 45: 2417-2424) and receptor binding assay (Gill, Lockey et al. 1986; Morgan, Swick et al. 2002). The preferred range of activity is 0.1 nM to 10 μM (the IC₅₀ of RU486 by the PR binding assay method is approximately 6 nM). Other suitable assays are described in the “Exemplification” section, below.

Preferred compounds that satisfy the criteria for use in the invention include RU486 (“mifepristone”, Roussel Uclaf, Paris; U.S. Pat. No. 4,386,085), its monodemethylated and didemethylated derivatives (RU42633 and RU42848), its alcoholic non-demethylated derivative (RU42698) and the demethyl and didemethyl derivatives of RU42698.

The structures of these compounds are shown below:

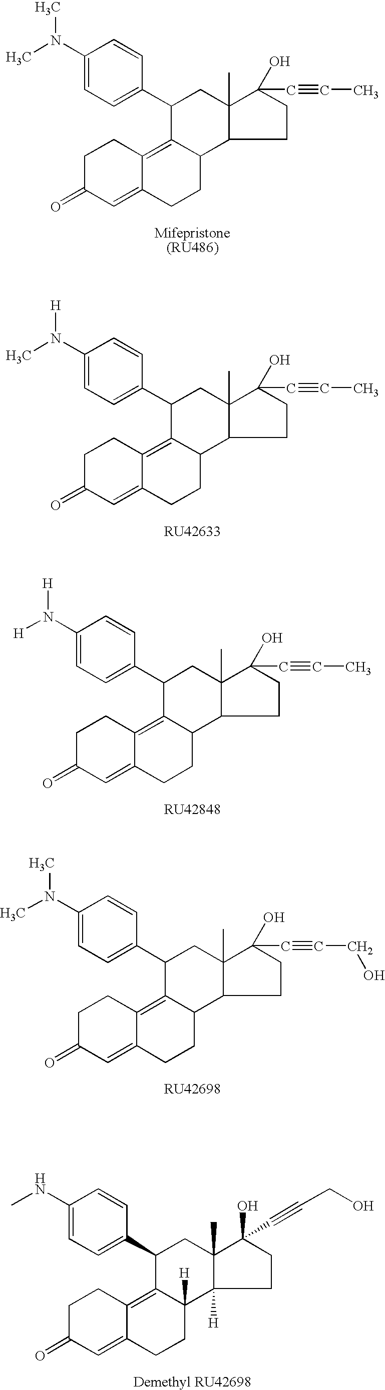

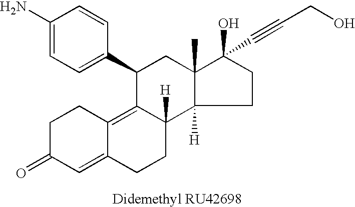

Other suitable compounds include onapristone (Schering AG, Berlin; U.S. Pat. No. 4,780,461), the structure of which is shown below:

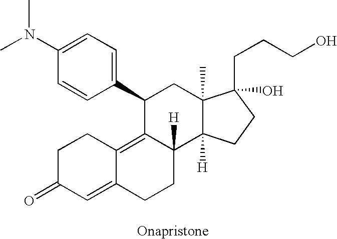

Other suitable progesterone antagonists include the steroids described in the following patents and patent applications: U.S. Pat. No. 4,609,651, especially the compound lilopristone (11-beta-(4-dimethylaminophenyl)-17-beta-hydroxy-17-alpha-(3-hydroxy-prop-1-(Z)-enzyl-4,9(10)-estradien-3-one); U.S. Pat. No. 5,089,635, especially the compounds 11-beta-(4-acetylphenyl)-17-beta-hydroxy-17-alpha-(1-propinyl)-4,9-estradien-3-one and 11-beta-(4-acetylphenyl)-17beta-hydroxy-17-alpha-(3-hydroxy-1(2)-propenyl)-4,9-estradien-3-one; U.S. Pat. No. 5,095,129; EP-A 04042831; and other anti-gestations, e.g., U.S. Pat. No. 4,891,368. Also, Faslodex ICI 182,780, which has antiprogestin activity (Wu, Liang et al. 2005), and compounds made by RW Johnson (Palmer, Campen et al. 2000): tetrahydropyridazines exemplified by RWJ 26819. Toripristone, ZK 112993, ZK 98299 (onapristone), ZK 98734, ZK 114043, ZK 114863 etc, aglepristone (RU534), RU43044, JNJ 1250132 (Allan, Palmer et al. 2006), lilopristone, SPRMs exemplified by asoprisnil (Schubert, Elger et al. 2005), ZK 230211 (Fuhrmann, Hess-Stumpp et al. 2000), RTI 3021-012 and RTI 3021-022 (Wagner, Pollio et al. 1999), CDB-2914 (Hild, Reel et al. 2000), CDB-4124 and CDB-4453 (Attardi, Burgenson et al. 2002), 16 alpha substituted RU486 (Wagner, Pollio et al. 1996) and compounds in papers (Giannoukos, Szapary et al. 2001; Leonhardt and Edwards 2002; Sathya, Jansen et al. 2002; Chabbert-Buffet, Meduri et al. 2005). Also included are 17-spirofuran-3′-ylidene steroids (U.S. Pat. No. 5,292,878) 11-arylsteroid compounds as disclosed in U.S. Pat. No. 4,921,845, dihydroquinolone (U.S. Pat. No. 5,688,810), cyclocarbamate derivatives as disclosed in U.S. Pat. No. 6,509,334, the PR ligands disclosed in the U.S. Pat. No. 5,693,646, U.S. Pat. No. 5,696,127, U.S. Pat. No. 5,994,544, U.S. Pat. No. 6,358,947, U.S. Pat. No. 6,713,478, U.S. Pat. No. 6,566,372, U.S. Pat. No. 6,436,929, U.S. Pat. No. 6,358,948, U.S. Pat. No. 7,081,457, the 5-(1′,1′-cycloalkyl/alkenyl)methylidene 1,2-dihydro-5H-chromeno[3,4-f]quinolines and 5-cycloalkenyl 5H-chromeno[3,4-f]quinolines as disclosed by Zhi, et al (U.S. Pat. Nos. 7,084,151 and 7,071,205). Also included are the selective progesterone receptor modulators disclosed in U.S. Pat. Nos. 7,084,151 and 7,071,205 and the PR antagonists described by Jones, et al., (Edwards, Zhi et al. 1998; Hamann, Winn et al. 1998; Tegley, Zhi et al. 1998; Zhi, Tegley et al. 1998; Zhi, Tegley et al. 1998; Zhi, Tegley et al. 1999; Zhi, Tegley et al. 2000; Zhi, Ringgenberg et al. 2003; Zhi, Tegley et al. 2003; Zhi, Tegley et al. 2003), the amide PR ligand described by Combs, et al., (Combs, Reese et al. 1995; Combs, Reese et al. 1995), the PR antagonists described by Hamann, et al., (Hamann, Farmer et al. 1995; Hamann, Farmer et al. 1996; Jones, Pathirana et al. 1996; Hamann, Winn et al. 1998) and the tatrahydronaphthofuranones and tetrahydrobenzindolones described by Kurihara, et al., (Kurihara, Shinei et al. 2006; Shinei, Kurihara et al. 2006).

Thus, included are the following compounds:

| Compound | Structure |
|---|---|
| Wyeth 6 | 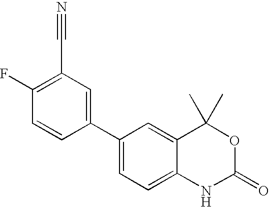 |
| Wyeth 4j | 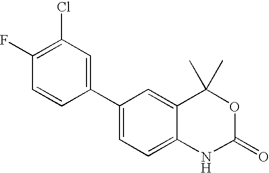 |
| Wyeth 4g | 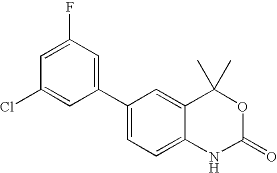 |
| Wyeth 4h | 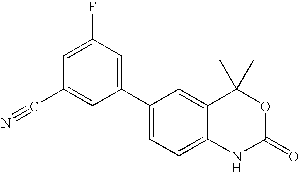 |
| LG120753 | 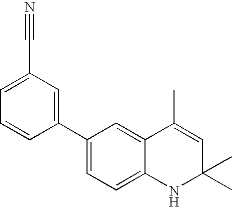 |
| Asoprisnil | 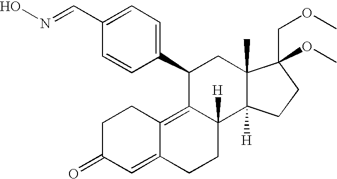 |
| LG121046 | 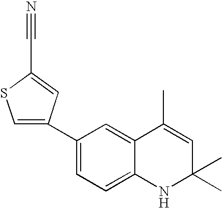 |
| Onapristone | 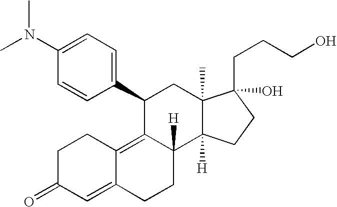 |

Other suitable compounds include those listed below:

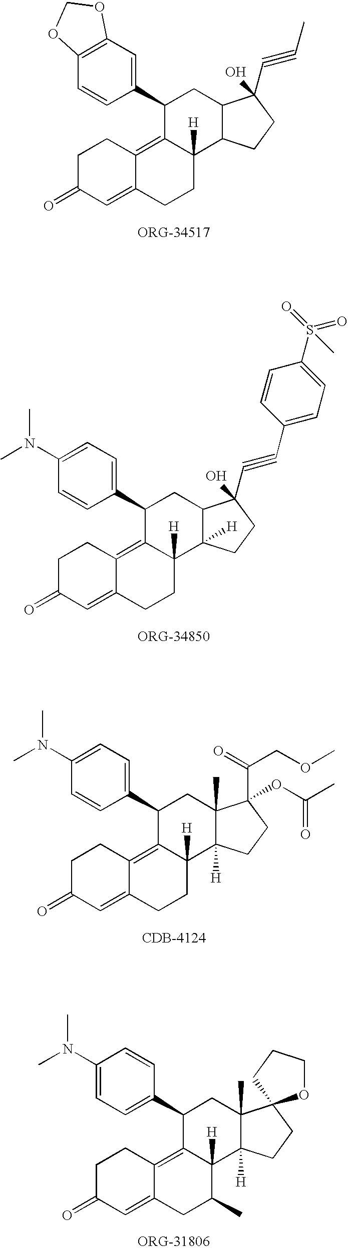

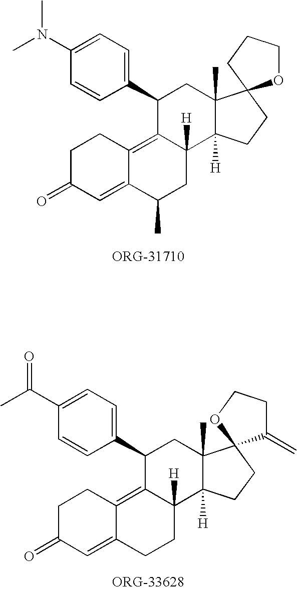

Other suitable compounds are described in WO99/25360, EP277676, EP289073, EP321010, EP549041 and EP582338 (the disclosure of which relating to progesterone antagonists is hereby incorporated herein by reference).

The invention can be used for the treatment of neovascularisation in age-related macular degeneration (AMD), including in particular wet AMD, ocular histoplasmosis syndrome, pathologic myopia, angioid streaks, idiopathic disorders, choroiditis, choroidal rupture, overlying choroid nevi, Best's disease, Stargardt's disease, Vogt-Koyanagi-Harada syndrome, toxoplasmosis, certain inflammatory diseases, sickle cell disease, diabetic retinopathy and other proliferative retinopathy, retinopathy of prematurity, neovascular glaucoma, sarcoidosis, syphilis, pseudoxanthoma elasticum, vein or artery occlusion, carotid obstructive disease, chronic uveitis/vitritis, mycobacterial infection, Lyme's disease, Eale's disease, systemic lupus erythomatosus, Behcet's disease, infections causing retinitis, optic pits, par planitis, chronic retinal detachment, hyperviscosity syndromes, capillary haemangioma including von Hippel-Lindau disease, trauma and post-laser complication.

An enhanced effect on retinal/choroidal/corneal NV and/or age-related macular degeneration may be achieved by co-administering a PR antagonist with another agent that is used for these conditions, such as macugen, lucentis, VEGF inhibitors, VEGF receptor tyrosine kinase inhibitors, protein kinase C inhibitors, inhibitors of other angiogenic proteins such as insulin-like growth factor and angiopoietin, recombinant angiostatic factors such as pigment epithelium-derived factor (PEDF), thrombospondin, and endostatin, somatostatin analogues, corticosteroids (such as but not limited to triamcinolone and anecortave acetate), statins (inhibitors of HMG-CoA reductase), squalamine lactate, thiamine and its analogues, angiotensin receptor blockers and rapamycin. The PR antagonist may also be administered as an adjunct to laser coagulation, photodynamic therapy (including visudyne), or cryotherapy.

### Formulation and Posology

In general, the active compound may be administered by known means, in any suitable formulation, by any suitable route. For oral administration, it is preferably formulated as a tablet, troche, lozenge, capsule, emulsion, syrup or elixir. A compound of this invention is preferably administered to the eye topically or by an ocular drug delivery system.

Compositions for oral administration include known pharmaceutical forms for such administration, for example lozenges, pastilles, dispersible tablets, powders or granules or as a liquid for spraying into the mouth. Compositions intended for oral use may be prepared according to any method known to the art for the manufacture of pharmaceutical compositions, and such compositions may contain one or more agents selected from the group consisting of sweetening agents, flavouring agents, colouring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example corn starch or alginic acid; binding agents, for example starch gelatin, acacia, microcrystalline cellulose or polyvinyl pyrrolidone; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time-delay material such as glyceryl monostearate or glyceryl distearate may be employed.

For oral administration, the composition may be in any form that will release the active agent, when held in the mouth, whether for a short time or for a matter of hours. It may be malleable and non-disintegrating, and/or chewable or dispersible. Preferred examples of such compositions are gums, as well as wafers and dispersible tablets (described above). A flavorant will typically be included. It is particularly desirable if the flavorant has mucolytic properties. An example of such a flavorant is menthol.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate, polyvinyl pyrrolidone, gum tragacanth and gum acacia; dispersing or wetting agents may be a naturally occurring phosphatide, for example lecithin, or condensation products of an alkylene oxide with fatty acids, for example polyoxyethylene stearate, or condensation products of ethylene oxide with long-chain aliphatic alcohols, for example heptadecaethyleneoxycetanol, or condensation products of ethylene oxide with partial esters derived from fatty acids, for example polyoxyethylene sorbitan monooleate. The aqueous suspensions may also contain one or more preservatives, for example ethyl or n-propyl p-hydroxybenzoate, one or more colouring agents, one or more flavouring agents, and one or more sweetening agents, such as sucrose or saccharin.

Oily suspensions may be formulated by suspending the active ingredient in a vegetable oil, for example arachis oil, olive oil, sesame oil or coconut oil, polyoxyethylene hydrogenated castor oil, fatty acids such as oleic acid, or in a mineral oil such as liquid paraffin or in other surfactants or detergents. The oily suspensions may contain a thickening agent, for example beeswax, hard paraffin or cetyl alcohol. Sweetening agents, such as those set forth above, and flavouring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, suspending agent and one or more preservatives. Suitable sweetening, flavouring and colouring agents may also be present.

Compositions for use in the invention may be formulated in a manner known to those skilled in the art so as to give a controlled release, for example rapid release or sustained release, of the compounds of the present invention. Pharmaceutically acceptable carriers suitable for use in such compositions are well known in the art. The compositions of the invention may contain 0.1-99% by weight of active compound. The compositions of the invention are generally prepared in unit dosage form. Preferably, a unit dose comprises the active ingredient in an amount of 0.001 to 100 mg. The excipients used in the preparation of these compositions are the excipients known in the art.

Appropriate dosage levels may be determined by any suitable method known to one skilled in the art. It will be understood, however, that the specific dose level for any particular patient will depend upon a variety of factors including the activity of the specific compound employed, the age, body weight, general health, sex, diet, time of administration, route of administration, rate of excretion, drug combination and the severity of the condition to be treated. Preferably, the active agent is administered at a frequency of 1 to 4 times per day.

The compositions may be formulated in a manner known to those skilled in the art so as to give adequate delivery to the back of the eye, which may be by regular dosing, such as with an eye drop, or may using a delivery system to give a controlled release, such as slow release, of the compounds in the present invention. Pharmaceutically acceptable carriers suitable for use in such compositions are well known in the art. The compositions of the inventions may contain 0.001-99% by weight of the active compound. The compositions of the invention are generally prepared in unit dosage form. Preferably, a unit dose comprises the active ingredient in an amount of 0.001 to 500 mg. The excipients used in preparation of these compositions are the excipients known in the art.

Appropriate dosage levels may be determined by any suitable method known to one skilled in the art. It will be understood, however, that the specific dosage level for any particular patient will depend on a variety of factors including age, body weight, general health, sex, diet, time of administration, route of administration, rate of excretion, drug combination and the severity of the complaint. Preferably the active compound is administered at a frequency of 1 to 4 times per day for topical administration, or less often if a drug delivery system is used. A typical daily dosage for topical treatment is a formulation containing 0.001-10% active ingredient.

The pharmaceutical compositions of the invention may be administered by any means known to those skilled in the art for treatment of eye diseases. They are preferably administered in the form of aqueous solutions, suspensions, or gels of the active compound in the form of drops of liquid, liquid washes, sprays or ointments, or gel. Alternatively the active compounds may be applied to the eye via liposomes or other ocular delivery system. The compositions contain the active materials in admixture with excipients suitable for the manufacture of aqueous solutions, suspensions and gels.

Components of the composition may be chosen from any of those used in or capable of being used in a pharmaceutical formulation, especially those designed for topical administration to the eye. A non-exclusive list of components includes preservatives, stabilizers, chelating agents, dyes, antibiotics, antimicrobials, and anti-fungal agents. Preservatives including, but not limited to, benzalkonium chloride, thimerosal, and phenyl mercuric nitrate may be used in a range between about 0.001 to 1 percent by weight. The compositions of the present invention may further comprise pharmaceutically acceptable carriers, excipients, gels, solutions, or diluents suitable for topical ophthalmic administration, and may include pharmaceutically acceptable polymeric suspension agents, solubilising agents, solvents and surfactants. Suitable carriers or excipients include, but are not limited to, calcium carbonate, calcium phosphate, various sugars (e.g. mannitol), starches, cellulose derivatives, gelatin, N-lauroylsarcosine, non-ionic surfactants, such as polysorbates, alcohols, such as propylene glycol and glycerol, and water soluble polymers such as polyethylene glycol. Polymeric suspension agents may be used, comprising one or more polymers, including dextrans, polyethylene glycols, polyvinylpyrrolidone, polysaccharide gels, Gelrite.®, tyloxapol, octoxynols, cellulosic polymers like hydroxypropyl methylcellulose (hypromellose), and carboxyl-containing polymers such as acrylic acid-containing polymers and vinyl-containing polymers (e.g. polyvinylpyrollidone). Particularly preferred polymers include polycarbophil, the DuraSite.®. polymeric delivery system (InSite Vision, Inc., Alameda, Calif.), and mucomimetic polymers (see, e.g., U.S. Pat. No. 5,932,572). Dispersing or wetting agents may be a naturally occurring phosphatide for example lecithin, or condensation products of an alkylene oxide with fatty acids, for example polyoxyethylene stearate, or condensation products of ethylene oxide with long chain aliphatic alcohols, for examples heptadecaethyleneoxycetanol or polyoxyethylene hydrogenated castor oil, or condensation products of ethylene oxide with partial esters derived from fatty acids, for example polyoxyethylene sorbitan monooleate. Other components may include a chelating agent such as disodium edetate, and complexing agents such as hydroxypropyl-beta-cyclodextrin. Antioxidants may be added to protect the active component from oxidation during storage. Examples of such antioxidants include vitamin E and analogues thereof, ascorbic acid, and butylated hydroxytoluene (BHT). The pH of the inventive compositions is preferably between about 6 and about 8, and may be adjusted for the particular compound(s) used. Purified water USP and various acids and bases suitable for ophthalmic use, or combinations of acids and bases, may be used for adjusting the pH of the compositions. Non-limiting examples of acids and bases include acetic acid, boric acid, citric acid, lactic acid, phosphoric acid, hydrochloric acid, sodium hydroxide, sodium phosphate, sodium borate, sodium citrate, sodium acetate, sodium lactate, and TRIS.

The osmotic pressure of the compositions may be adjusted by methods known in the art to be between about 40 to about 400 milliosmolar (mOsM), more preferably between about 100 to about 300 mOsM. A preferred method of adjusting osmotic pressure is the addition of physiologically and ophthalmically acceptable salts. Sodium chloride, which approximates physiological fluid, is the preferred salt, for use in concentrations ranging from about 0.01 to about 1 percent by weight, or any value in that range. Preferably, the concentration is between about 0.1 to about 1 percent. Equivalent amounts of one or more salts made up of cations such as potassium, ammonium and the like and anions such as chloride, citrate, ascorbate, borate, phosphate, bicarbonate, sulfate, thiosulfate, bisulfite and the like, e.g., potassium chloride, sodium thiosulfate, sodium bisulfite, ammonium sulfate, and the like, can also be used in addition to or instead of sodium chloride to achieve osmotic pressures within the above-stated ranges. An example of a preferred composition is for an ocular solution containing the compound in aqueous solution with hydroxypropyl-beta-cyclodextrin, polyethylene glycol 400, and hydroxypropylmethylcellulose.

Examples of treatment scenarios envisaged for PR antagonism are as follows: (a) topical administration to the eye of RU486, or RU486 metabolite (RU42698, RU42848 and RU42633) or selective PR antagonist to individuals with CNV; (b) administration using an ocular drug delivery system of RU486, or RU486 metabolite (RU42698, RU42848 and RU42633) or selective PR antagonist to individuals with CNV; (c) topical administration to the eye of RU486, or RU486 metabolite (RU42698, RU42848 and RU42633) or selective PR antagonist to individuals with drusen characteristic of early age-related macular degeneration to reduce the risk of developing CNV; (d) administration using an ocular drug delivery system of RU486, or RU486 metabolite (RU42698, RU42848 and RU42633) or selective PR antagonist to individuals with drusen characteristic of early age-related macular degeneration to reduce the risk of developing CNV; (e) topical administration to the eye of RU486, or RU486 metabolite (RU42698, RU42848 and RU42633) or selective PR antagonist to individuals with retinal neovascularization; (f) administration using an ocular drug delivery system of RU486, or RU486 metabolite (RU42698, RU42848 and RU42633) or selective PR antagonist to individuals with retinal neovascularization; (g) topical administration to the eye of RU486, or RU486 metabolite (RU42698, RU42848 and RU42633) or selective PR antagonist to individuals with corneal neovascularization; (h) administration using an ocular drug delivery system of RU486, or RU486 metabolite (RU42698, RU42848 and RU42633) or selective PR antagonist to individuals with corneal neovascularization; (i) topical administration to the eye of any of the above compounds possessing PR antagonist activity given in combination with other agents that reduce the activity of VEGF (e.g., VEGF inhibitor, VEGF receptor tyrosine kinase inhibitors), protein kinase C inhibitors, inhibitors of other angiogenic proteins such as insulin-like growth factor and angiopoietin, recombinant angiostatic factors such as pigment epithelium-derived factor (PEDF), thrombospondin, endostatin, corticosteroids (such as but not limited to triamcinolone and anecortave) and statins (inhibitors of HMG-CoA reductase). These other agents may be given topically to the eye or introduced directly into the eye via injections or implant devices, or systemically.

### Exemplification

The invention will now be described with reference to specific studies. These are merely exemplary and for illustrative purposes only: they are not intended to be limiting in any way to the scope of the monopoly claimed or to the invention described. The studies provide evidence of utility of the present invention.

### Zebrafish Angiogenesis Assays

Zebrafish (Danio rerio) are ideal organisms for the investigation of blood vessel growth because they are transparent in the embryonic and larval stages of development so that blood vessels marked with a fluorescent marker can clearly be seen in vivo in the live animal. Zebrafish blood vessel development is well characterised and vessels can be visualised by microangiography or by expressing green fluorescent protein in blood vessels (Isogai et al. (2001) Dev Biol 230(2): 278-301; Covassin et al. (2006) Proc Natl Acad Sci USA 103(17): 6554-9). Zebrafish are also ideal vertebrate organisms in which to screen for active small molecules because the larvae can be treated with compound simply by immersion of the fish in compound solution in wells of a standard multi-well plate (12, 24 or 96-wells).

Zebrafish protein sequences bear remarkable similarity to the cognate human protein sequences, especially across important functional domains (Goldsmith (2004) Curr Opin Pharmacol 4(5): 504-12; Peterson at al. (2004) Nat Biotechnol 22(5): 595-9). In particular, zebrafish contain a progesterone receptor gene (NCBI Accession Number AAY85275.1) which is 67% identical and 96% similar to the human progesterone receptor over the ligand binding domain. Moreover, the VEGF signalling pathway is highly conserved between zebrafish and mammals (Covassin et al. (2006) Proc Natl Acad Sci USA 103(17): 6554-9). Thus, compounds that inhibit zebrafish blood vessel formation are likely to be anti-angiogenic in man.

Expression of the zebrafish PR can be detected as early as 48 hours post fertilisation and an assay was therefore developed to investigate the effects of compounds on blood vessel formation in zebrafish after this time. Previous studies on inhibition of blood vessel growth have focussed on formation of intersegmental vessels (Serbedzija et al. (1999) Angiogenesis 3(4): 353-9; Parng at al. (2002) Assay Drug Dev Technol 1(1 Pt 1): 41-8) but these particular vessels are specified and begin to form prior to the time when the PR is expressed in the zebrafish embryo. Accordingly, later-developing vessels around the gut, the vascular plexus of the mid gut which branches from the subintestinal vein and supraintestinal artery, were monitored.

### Methods

Methods have been developed for visualisation of zebrafish blood vessels by expression of green fluorescent protein in the vessels of transgenic fish. The lines of transgenic fish most frequently used for this are those expressing green fluorescent protein (GFP) under control of the zebrafish flit promoter (Lawson and Weinstein (2002) Dev Biol 248(2): 307-18) or flk1 promoter (Jin et al. (2005) Development 132(23): 5199-209).

A similar method was developed for screening of compounds using a line of fish that express GFP under control of an enhancer that drives expression in blood vessels, spinal cord and eyes. The fish are a cross between the et19.1 enhancer:Gal4 line (Scott et al. (2007) Nat Methods 4(4): 323-6) and a UAS:GFP line made in-house in a TL zebrafish background. Transgenic zebrafish embryos or larvae expressing GFP in the blood vessel walls were treated with test compounds by immersion in aqueous medium containing the compound at the required concentration, for a period of one or more days. At the end of the incubation period the fish were immobilised on their sides and the organisation and structure of the vasculature was visualised by fluorescent microscopy, which is possible because of the transparency of zebrafish larvae. The vessels can be identified by comparison with the online Interactive Atlas of Zebrafish Vascular Anatomy (Isogai et al. (2001) Dev Biol 230(2): 278-301).

Vessels such as the dorsal aorta, posterior cardinal vein, caudal artery, caudal vein, intersegmental vessels, dorsal longitudinal anastomotic vessels and the gut vessel plexus were assessed in each fish and compared to the control group. The gut vessel plexus formed during the time at which progesterone receptor is expressed and these were the vessels that were shown to be inhibited by progesterone receptor antagonists. Development of these vessels was shown to be VEGF dependent because vessel formation was inhibited by incubation with VEGF receptor inhibitor SU5416. A subjective score was given to the fore/mid gut vessel plexus (by assessment of the number of visible vessels and extent of spread of the plexus), ranging from “3” for a normal extensively vascularised gut, to “0” for a complete absence of any branching of vessels around the gut. The intestinal vessel plexus forms between 3 and 5 days post fertilisation (d.p.f.) and so these assays were usually scored at 5 d.p.f. The vessels could also be quantified by digital photography and quantification of fluorescence levels or number of vessels in the region of interest.

Those skilled in the art will appreciate that other methods for visualisation of blood vessels may be used, including: (1) fluorescent angiography (injection of a fluorescent marker molecule such as FITC-dextran into the vascular system in live fish and visualisation of the vessels by fluorescent microscopy), using methods such as those described by (Weinstein et al. (1995) Nat Med 1(11): 1143-7; Peterson et al., (2004) Nat Biotechnol 22(5): 595-9); (2) staining of vessels with antibody specific for blood vessels (such as anti-Tie-2 or anti-VEGF receptor antibody); and (3) staining for endogenous alkaline phosphatase activity (Habeck et al., (2002) Curr Biol 12(16): 1405-12; Serbedzija et al. (1999) Angiogenesis 3(4): 353-9; Parng et al. (2002) Assay Drug Dev Technol 1(1 Pt 1): 41-8). It is also to be understood that an alternative to assessing developmental angiogenesis (occurring in embryos/larvae) is the assessment of angiogenesis in adult fish, such as during regeneration of the tail fin. Suitable methods for use in such assays are described for example by Bayliss et al. (2006) Nat Chem Biol 2(5): 265-73). Angiogenesis can be scored by using gene expression as a surrogate, for example by quantification of mRNA levels for genes involved in angiogenesis (e.g. VEGF and its receptors). Suitable methods for use in such assays include RT-PCR or RNA in situ hybridization or quantification of protein (using methods such as Western blotting or ELISA).

It will also be appreciated that compounds may also be tested in cell culture assays in vitro using mammalian endothelial cells, in particular a tubule development assay in normal human endothelial cells co-cultured with interstitial cells, as disclosed in U.S. Pat. No. 6,225,118, GB2331763 and EP1023599. These cultures spontaneously develop a network of capillary-like tubules after 10-14 days in culture. Tubule development is believed to closely mimic in vivo angiogenesis and is enhanced by known stimulators of angiogenesis and suppressed by known angiogenesis inhibitors, both in a dose-dependent manner (Bishop et al., (1999) Angiogenesis 3(4): 335-44; Donovan et al. (2001) Angiogenesis 4(2): 113-21; Clarke et al. (2006) Cancer Res 66(7): 3504-12). Other suitable in vitro assays include assessment of proliferation, migration, invasion and wound healing in endothelial cell cultures, and a rat aortic ring culture assay.

### Mammalian Angiogenesis Assays

Those skilled in the art will also appreciate that compounds for use according to the invention can also be identified using mammalian angiogenesis assays. In particular, compounds suitable for use in the treatment of wet AMD can be identified using assays based on inhibition of laser-induced choroidal neovascularisation in mammalian models (such as the laser-induced CNV model). This involves laser photocoagulation of the RPE and choroid, resulting in a wound response that includes formation of a neovascular lesion. This is usually performed in rodents or primates. The protocol used is essentially as described by Kinose et al. (2005) Mol Vis 11: 366-73). Methods for assessment of neovascular lesions include angiography with fluorescein or indocyanine green, detected as regions of leakage from vessels by fundus photography using an instrument such as a confocal scanning laser opthalmoscope (see e.g. Takehana et al. (1999) Invest Opthalmol Vis Sci 40(2): 459-66; Koh et al., (2004) Invest Opthalmol Vis Sci 45(2): 635-40). Also, neovessels can be detected by ex vivo analysis of flatmounts after infusion with FITC dextran or similar agent (Edelman and Castro (2000) Exp Eye Res 71(5): 523-33), or by staining with a specific antibody, histochemical visualisation (Semkova et al. (2003) Invest Opthalmol Vis Sci 44(12): 5349-54) or by histological analysis of stained sections of retinal tissue (Takehana et al., (1999) Invest Opthalmol Vis Sci 40(2): 459-66; Yanagi et al. (2003) Invest Opthalmol Vis Sci 44(2): 751-4; Koh et al. (2004) Invest Opthalmol Vis Sci 45(2): 635-40). Other suitable in vivo angiogenesis assays for measurement of inhibition of angiogenesis at other sites include retinal and corneal angiogenesis models in rodents and rabbits and mammalian models that involve insertion into various regions of the body of an implant containing angiogenic factors such as VEGF or basement membrane extracts.

### Results

Assays using Tg 043/050 uas:GFP+/−enhancer:gal4+/−×TL (“panretinal” enhancer) fish, incubated with various compounds (see Table below) from 24 h.p.f. to 5 d.p.f. (unless otherwise stated), then scoring the extent of the gut vessel plexus by microscopy. A score of 3 represents a good, fully formed gut vessel plexus, with 20 or more branched vessels; 2 represents as slightly less well formed plexus with 10-19 vessels; 1 represents a poorly formed plexus with 1-9 vessels; 0 represents a complete absence of visible gut vessels. Each of the compounds was tested in a 24 hour MTC assay in the TL strain of wild type fish (24-48 h.p.f.). The MTC from this assay was used as the highest concentration for assays in the transgenic fish, with half-log intervals.

RU486 inhibition was dose-dependent (data not shown) and was rescued by progesterone (10 μM of each), suggesting that the effects of RU486 in this system are regulated by progesterone receptor. RU42698 also showed concentration-dependent inhibition of vessels at 30-100 μM. RU42633 concentration response curves (1-5 dpf) showed clear concentration-dependent inhibition of gut vessels with best inhibition at 30 μM. Wyeth 4h also showed concentration-dependent inhibition, maximal at 30 μM (data not shown). LG120753 showed concentration-dependent inhibition at 0.3-3 μM.

| | | | concs | | Effective |
|---|---|---|---|---|---|
| | MTC | No. of | scored | Inhibition of gut | concs |
| Compound | (μM) | assays | (μM) | vessel formation | (μM) |
| RU486 | 10-30 | 3 | 0.3-10 | +++ | 3-10 |
| RU42633 | 30 | 2 | 0.3-30 | +++ | 3-10 |
| RU42698 | 100 | 3 | 1-100 | ++ | 30-100 |
| RU42848 | 100 | 2 | 1-100 | + | (10-30) |
| Wyeth 6 | 100 | 3 | 1-100 | + | (10), 30 |
| Wyeth 4j | 1-3 | 3 | 0.1-3 | + | 1-3 |
| Wyeth 4g | 3 | 2 | 0.1-3 | ++ | 0.3, 3 |
| Wyeth 4h | 30 | 2 | 0.3-30 | ++ | 1-30 |
| LG120753 | 3 | 2 | 0.03-3 | + | 1-3 |
| Asoprisnil | 10 | 2 | 0.3-30 | + | 10 |
| LG121046 | 3-10 | 2 | 0.1-10 | ++ | 1-10 |

Key to extent of inhibition:
+++ = inhibition of vessels equivalent to that of 10 μM RU486 (which has a mean score of 1 to 1.5).
++ = inhibition of vessels but to a lesser extent that RU486.
+ = slightly fewer vessels than in controls but much less inhibition than with RU486.
− = no detectable inhibition.

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

### REFERENCES

Allan et al. (2006). Steroids 71(7): 578-84.
Attardi et al. (2002). Mol Cell Endocrinol 188(1-2): 111-23.
Baulieu and Segal (1985). The antiprogestin steroid RU486 and human fertility control. New York, Plenum Press.
Baulieu, E. E. and A. Ulmann (1986). Hum Reprod 1(2): 107-10.
Bressler, N. M., S. B. Bressler, et al. (1988). Surv Opthalmol 32(6): 375-413.
Chabbert-Buffet, N., G. Meduri, et al. (2005). Hum Reprod Update 11(3): 293-307.
Cheeks, L. and K. Green (1986). Curr Eye Res 5(9): 705-9.
Combs, D. W., K. Reese, et al. (1995). J Med Chem 38(25): 4880-4.
Combs, D. W., K. Reese, et al. (1995). J Med Chem 38(25): 4878-9.
DeBattista, C. and J. Belanoff (2005). Curr Alzheimer Res 2(2): 125-9.
Edwards, D. P., M. Altmann, et al. (1995). J Steroid Biochem Mol Biol 53(1-6): 449-58.
Edwards, J. P., L. Zhi, et al. (1998). J Med Chem 41(15): 2779-85.
Eisinger, S. H., T. Bonfiglio, et al. (2005). J Minim Invasive Gynecol 12(3): 227-33.
Fiscella, K., S. H. Eisinger, et al. (2006). Obstet Gynecol 108(6): 1381-7.
Flores, B. H., H. Kenna, et al. (2006). Neuropsychopharmacology 31(3): 628-36.
Fuchsjager-Mayrl, G., J. Nepp, et al. (2002). Invest Opthalmol Vis Sci 43(9): 2841-4.
Fuhrmann, U., H. Hess-Stumpp, et al. (2000). J Med Chem 43(26): 5010-6.
Gagne, D., M. Pons, et al. (1985). J Steroid Biochem 23(3): 247-51.
Gallagher, P., S. Watson, et al. (2005). Biol Psychiatry 57(2): 155-61.
Giannoukos, G., D. Szapary, et al. (2001). Mol Endocrinol 15(2): 255-70.
Gill, J. C., P. M. Lockey, et al. (1986). J Med Chem 29(8): 1537-40.
Hamann, L. G., L. J. Farmer, et al. (1996). J Med Chem 39(9): 1778-89.
Hamann, L. G., L. J. Farmer, et al. (1995). Ann NY Acad Sci 761: 383-7.
Hamann, L. G., D. T. Winn, et al. (1998). Bloom Med Chem Lett 8(19): 2731-6.
Hild, S. A., J. R. Reel, et al. (2000). Hum Reprod 15(4): 822-9.
Jones, T. K., C. Pathirana, et al. (1996). J Steroid Biochem Mol Blot 56(1-6 Spec No): 61-6.
Kaschula, C. H., M. H. Jin, et al. (2006). Exp Eye Res 82(1): 111-21.
Kettel, L. M., A. A. Murphy, et al. (1996). Fertil Steril 65(1): 23-8.
Klein, B. E. and R. Klein (1982). Arch Opthalmol 100(4): 571-3.
Kompella, U. B., N. Bandi, et al. (2003). Invest Opthalmol Vis Sci 44(3): 1192-201.
Kurihara, K., R. Shinei, et al. (2006). Bloom Med Chem 14(14): 4862-78.
Lanthier, A. and V. V. Patwardhan (1988). Brain Res 463(2): 403-6.
Leibowitz, H. M., D. E. Krueger, et al. (1980). Surv Opthalmol 24(Suppl): 335-610.
Leonhardt, S. A. and D. P. Edwards (2002). Exp Biol Med (Maywood) 227(11): 969-80.
Li, Y. C., S. Hayes, et al. (1997). J Mol Neurosci 8(2): 145-58.
Lindsey, J. D. and R. N. Weinreb (1994). Invest Opthalmol Vis Sci 35(10): 3640-8.
Lippman, M. E., B. O. Wiggert, et al. (1974). J Biol Chem 249(18): 5916-7.
Moguilewsky, M. and D. Philibert (1984). J Steroid Biochem 20(1): 271-6.
Morgan, B. P., A. G. Swick, et al. (2002). J Med Chem 45(12): 2417-24.
Muranaka, K., Y. Yanagi, et al. (2005). FEBS Lett 579(27): 6084-8.
Palmer, S., C. A. Campen, et al. (2000). J Steroid Biochem Mol Biol 75(1): 33-42.
Perrault, D., E. A. Eisenhauer, et al. (1996). J Clin Oncol 14(10): 2709-12.
Phillips, C. I., K. Green, et al. (1984). Lancet 1(8380): 767-8.
Ross, A. C. (1982). J Biol Chem 257(5): 2453-9.
Sathya, G., M. S. Jansen, et al. (2002). Endocrinology 143(8): 3071-82.
Schlingemann, R. O. (2004). Graefes Arch Clin Exp Opthalmol 242(1): 91-101.
Schubert, G., W. Elger, et al. (2005). Semin Reprod Med 23(1): 58-73.
Shinei, R., K. Kurihara, et al. (2006). Bioorg Med Chem 14(14): 4850-61.
Simpson, G. M., A. El Sheshai, et al. (2005). J Clin Psychiatry 66(5): 598-602.
Sitruk-Ware, R. and I. M. Spitz (2003). Contraception 68(6): 409-20.
Spitz, I. M., S. M. Grunberg, et al. (2005). Fertil Steril 84(6): 1719-26.
Sultana, Y., R. Jain, et al. (2006). Curr Drug Deliv 3(2): 207-17.
Suzuki, T., Y. Kinoshita, et al. (2001). Curr Eye Res 22(1): 28-33.
Tegley, C. M., L. Zhi, et al. (1998). J Med Chem 41(22): 4354-9.
Tsukahara, S., T. Sasaki, et al. (1986). Br J Opthalmol 70(6): 451-5.
Vinores, S. A., W. H. Xiao, et al. (2006). J Cell Physiol 206(3): 749-58.
Wagner, B. L., G. Pollio, et al. (1999). Endocrinology 140(3): 1449-58.
Wagner, B. L., G. Pollio, et al. (1996). Proc Natl Acad Sci USA 93(16): 8739-44.
Wickham, L. A., J. Gao, et al. (2000). Acta Opthalmol Scand 78(2): 146-53.
Wu, J., Y. Liang, et al. (2005). Int J Oncol 27(6): 1647-59.
Yeoh, J., J. Sims, et al. (2006). Expert Opin Pharmacother 7(17): 2355-68.
Zhi, L., J. D. Ringgenberg, et al. (2003). Bioorg Med Chem Lett 13(12): 2075-8.
Zhi, L., C. M. Tegley, et al. (1998). Bioorg Med Chem Lett 8(23): 3365-70.
Zhi, L., C. M. Tegley, et al. (1998). J Med Chem 41(3): 291-302.
Zhi, L., C. M. Tegley, et al. (1999). J Med Chem 42(8): 1466-72.
Zhi, L., C. M. Tegley, et al. (2003). Bioorg Med Chem Lett 13(12): 2071-4.
Zhi, L., C. M. Tegley, et al. (2003). J Med Chem 46(19): 4104-12.
Zhi, L., C. M. Tegley, et al. (2000). Bioorg Med Chem Lett 10(5): 415-8.

1. A method for treating a condition associated with blood vessel formation wherein said method comprises administering to a subject in need of such treatment an agent having progesterone antagonist properties.

2. The method, according to claim 1, wherein the condition is an ocular condition.

3. The method, according to claim 2, for the treatment for an ocular condition in which neovascularisation is involved.

4. The method, according to claim 1, wherein the condition is age-related macular degeneration, choroidal neovascularisation, retinal neovascularisation, or corneal neovascularisation.

5-7. (canceled)

8. The method, according to claim 1, wherein the condition is selected from ocular histoplasmosis syndrome, pathologic myopia, angioid streaks, idiopathic disorders, choroiditis, choroidal rupture, overlying choroid nevi, Best's disease, Stargardt's disease, Vogt-Koyanagi-Harada syndrome, toxoplasmosis, sickle cell disease, diabetic retinopathy and other proliferative retinopathy, retinopathy of prematurity, neovascular glaucoma, sarcoidosis, syphilis, pseudoxanthoma elasticum, vein or artery occlusion, carotid obstructive disease, chronic uveitis/vitritis, mycobacterial infection, Lyme's disease, Eale's disease, systemic lupus erythematosus, Behcet's disease, infections causing retinitis, optic pits, par planitis, chronic retinal detachment, hyperviscosity syndromes, capillary haemangioma including von Hippel-Lindau disease, trauma, post-laser complication, epidemic keratoconjunctivitis, Vitamin A deficiency, contact lens overwear, atopic keratitis, superior limbic keratitis, pterygium keratitis sicca, sjogrens, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi sarcoma, Mooren ulcer, Terrien's marginal degeneration, marginal keratolysis, rheumatoid arthritis, systemic lupus, polyarteritis, trauma, Wegeners sarcoidosis, Scleritis, Steven's Johnson disease, pemphigoid radial keratotomy, and corneal graft rejection.

9. The method, according to claim 1, wherein the agent is a progesterone receptor antagonist.

10. The method, according to claim 1, wherein the agent has a progesterone receptor binding activity of 0.01 nM to 10 μM.

11. The method, according to claim 1, wherein the agent is mifepristone, onapristone or asoprisnil.

12. The method, according to claim 1, wherein the agent is a metabolite of mifepristone selected from RU42698, RU 42848, RU42633 and the demethyl and didemethyl derivatives of RU42698.

13. The method, according to claim 1, wherein the medicament is administered orally or topically.

14. The method, according to claim 13, wherein the medicament is administered topically to the eye.

15. The method, according to claim 14, wherein the medicament is an eye drop.

16. The method, according to claim 1, wherein the medicament comprises a slow release drug delivery system.

17. The method, according to claim 1, wherein the subject of treatment is also given, topically, systematically or directly into the eye via injection or implant, another drug selected from macugen, Lucentis, avastin and other VEGF inhibitors, VEGF receptor tyrosine kinase inhibitors, protein kinase C inhibitors, inhibitors of other angiogenic proteins, recombinant angiostatic factors, somatostatin analogues, corticosteroids, statins (inhibitors of HMG-CoA reductase), squalamine lactate, thiamine and its analogues, angiotensin receptor blockers and rapamycin.

18. A method for treating wet AMD wherein said method comprises administering, to a subject in need of such treatment, mifepristone (RU486), or a metabolite or pharmaceutically acceptable derivative thereof.

19. The method of claim 18 wherein the mifepristone (RU486) metabolite is selected from RU42698, RU42848, RU42633 and the demethyl and didemethyl derivatives of RU42698.

20. A pharmaceutical composition for topical administration to the eye comprising mifepristone (RU486), or a metabolite or pharmaceutically acceptable derivative thereof.

21. The pharmaceutical composition of claim 20 wherein the mifepristone (RU486) metabolite is selected from RU42698, RU42848, RU42633 and the demethyl and didemethyl derivatives of RU42698.

22-29. (canceled)

30. The method of claim 18, wherein the subject treated is also given, topically, systematically or directly into the eye via injection or implant, another drug selected from macugen, Lucentis, avastin and other VEGF inhibitors, VEGF receptor tyrosine kinase inhibitors, protein kinase C inhibitors, inhibitors of other angiogenic proteins, recombinant angiostatic factors, somatostatin analogues, corticosteroids, statins (inhibitors of HMG-CoA reductase), squalamine lactate, thiamine and its analogues, angiotensin receptor blockers and rapamycin.
